(19) United States
(12) Patent Application Publication
(10) Pub. No.: US 2009/0204002 A1
(43) Pub. Date: Aug. 13, 2009

(54) Use of signal strength indicator in ultrasound fetal monitors

(76) Inventor: Ashit M. Pandit, Laurel, MD (US)

Correspondence Address: ROGER M. RATHBUN, 13 MARGARITA COURT, HILTON HEAD ISLAND, SC, 29926, US

(21) Appl. No.: 12/069,648

(22) Filed: Feb. 12, 2008

(51) Int. Cl. A61B 8/14

(52) U.S. Cl. 600/453

(57) ABSTRACT

A method and system for positioning the transducer of a Doppler ultrasound fetal heat rate monitor at an optimum location on the abdomen of the pregnant patient. The method entails using a transducer positioned on the abdomen of a pregnant patient. Use is made of the conventional audio envelope that drives the audio amplifiers of a Doppler ultrasound monitor. That audio envelope is used to determine an average maximum and minimum values of the signal strength over a period of time. Those values establish a full span and can be continuously updated as the monitoring is carried out. The average maximum and minimum strength values are used to create a visual display of a particular set of signals from the patient for use by the caregiver to locate the transducer at a optimum location contacting the skin of the patient in the abdominal area.

### FIELD OF THE INVENTION

The present invention relates to a system for determining the optimum position of transducers in ultrasound fetal monitors, and, more particularly, to the use of a signal strength indicator to position the ultrasound transducers at the optimum location on the patient.

### BACKGROUND OF THE INVENTION

One of the common means for monitoring fetal heart rate in a labor and delivery room is through the user of Doppler ultrasound. In such systems, ultrasonic waves are generated by means of a transducer that is placed against the skin of the patient and the ultrasonic waves are directed toward the fetus where they are returned to the same transducer. The return echoes carry information obtained by Doppler shift so as to provide the caregiver with the heart rate of the fetus and that information can be displayed on a heart rate monitor.

One of the difficulties experienced with current monitors using Doppler ultrasound, however, is that fetal movements often result in a loss of the echo signals that are conveyed to and thus detected by the transducer and which results in a loss of the monitoring function. In such case, the caregiver typically has to reposition the transducer in order to continue with the monitoring.

One current method of carrying out the re-positioning to recapture the fetal heart rate involves moving the transducer across the skin of the patient and listening for the strongest audio signal from the monitor. However, the ultrasonic audio is loud and often disturbs the sleep of the pregnant patient.

Accordingly, it would be advantageous to have a system and a method that could allow the caregiver to carry out the repositioning of the transducer after loss of a signal that is quiet and does not rely on the use of an audio signal so that the patient is not disturbed.

### BRIEF SUMMARY OF THE INVENTION

Therefore, with the present invention, there is provided a method and system for positioning an ultrasound transducer of a Doppler ultrasound monitor along and in contact with the skin of a patient in the abdominal area.

With the present invention, use is made of the conventional audio envelope that drives the audio amplifiers of a Doppler ultrasound monitor. With the invention, average maximum and minimum values of the signal strength are determined over a period of time. Those values establish a full span and can be continuously updated as the monitoring is carried out. By thus having the average maximum and minimum strength values, any particular signal strength of the audio envelope can then be expressed and displayed visually as a percentage of the full scale envelope.

As such the caregiver can visually monitor the display indicating signal strength in order to find the appropriate location for the transducer contacting the abdomen of the patient.

Accordingly, in the method aspect of the present invention, the transducer is positioned on the skin of the patient and an ultrasound signal is directed inwardly within the patient toward the fetus therein and an echo signal is received by the transducer. The strength of the return signal is monitored and the transducer is repositioned in accordance with that signal strength, that is, the transducer can be moved to a location where there is a strong signal received by the transducer.

In an exemplary embodiment, the echo or return signals are processed to calculate an average maximum level and an average minimum level so as to provide a full scale of the audio envelope of the signals from the patient. A percentage of the full scale envelope can then be used to indicate the strength of a particular signal received by the transducer.

In the system aspect of the present invention, there is a system for introducing the ultrasound signals into the patient and a receiver for receiving back the echo signals and a signal strength indicator that provides a visual indication of the strength of the return signal received by the transducer. The signal strength is used as a visual indication to a caregiver as to the location of a good strength signal for the location of the transducer. The visual display can be readily available within the sight of the caregiver for continual monitoring that display and can be, among other locations, located on the heart rate monitor or on the transducer itself.

These and other features and advantages of the present invention will become more readily apparent during the following detailed description taken in conjunction with the drawings herein.

### BRIEF DESCRIPTION OF THE DRAWINGS

FIG. 1 is a schematic view of the fetal heat rate monitor system using the present invention; and

FIG. 2 is a flow chart illustrating the method of the present invention.

### DETAILED DESCRIPTION OF THE INVENTION

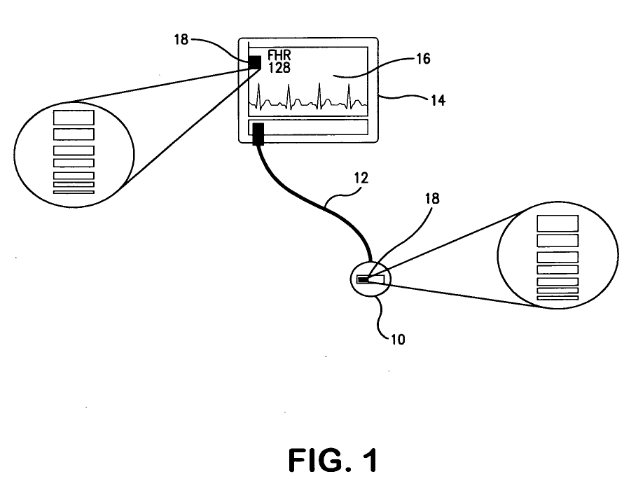

Referring now to FIG. 1, there is shown a schematic view of the present invention and wherein there is a transducer 10 that is adapted to be positioned in contact with the abdomen of the pregnant patient. As is conventional, the transducer 10 directs ultrasound signals into the womb of the patient such that the ultrasound signals echo off of the fetus to produce return or echo signals that are received by the transducer 10 with a Doppler shift indicative of the heat rate of the fetus.

Those echo signals from the fetus are sent, via a cable 12, to a fetal heart rate monitor 14 that includes a display 16 so that a caregiver can visually perceive and continually monitor the fetal heart rate. As is also conventional, the return audio echo signals form an ultrasound audio envelope within which the audio signals lie.

As therefore can be seen, there is also a signal strength indicator 18, preferably in the form of a series of bars, and which, in accordance with the present invention, provides a visual indication of the strength of the return echo signals that are received by the transducer 10. The signal strength indicator 18 can be located as a part of the display 16 of the fetal heart rate monitor 14 or can be incorporated into the transducer 10.

In either event, the caregiver is provided with a visual indication of the signal strength and thus a means by which the caregiver can relocate the transducer 10 to a location along the abdomen of the patient where the signal strength is sufficiently strong.

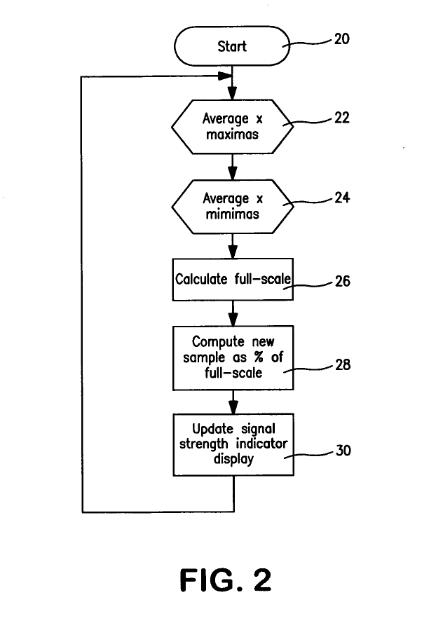

Turning now to FIG. 2, there is shown a flow chart illustrating the steps carried out with the method of the present invention. Accordingly, the method is initialized at the start block 20. In block 22, there is a averaging step where the audio envelope that is conventionally produced by a Doppler ultrasound fetal heart rate monitor is monitored such that, at block 12, the maximum audio signals that are produced in the creation of the envelope are averaged to produce an average maximum strength of the audio signals. In block 24, the same type of averaging step is carried out, however, in block 24, the minimum signals are averaged to calculate an average minimum signal. Thus, at this point, the method has taken the conventional audio envelope and calculated an average maximum strength signal and an average minimum strength signal.

Next, at block 26, having the average maximum and minimum signals, there is created a full scale for the signal strength of the echo signals from the fetus and received by the transducer. The average maximum and minimum then set the range of the strength bars, as shown in the signal strength indicator 18 of FIG. 1.

Accordingly, as each new audio signal is received by the transducer 10, in block 28, the strength of that signal can be related to a position along the bars and the caregiver can get a visual indication of the relative strength of the real time signal as related to the average minimum and maximum audio signals in the form of the number of bars.

Finally in block 30, the method returns to the initial averaging steps so the signal strength indicator 18 is continuously updated.

Thus, by the use of the signal strength indicator, the caregiver has a visual, non-audio, indicator that allows the positioning of the transducer along the abdomen of the patient in accordance with a strong signal without the loud noise that can disturb the pregnant patient.

Those skilled in the art will readily recognize numerous adaptations and modifications which can be made to the of the present invention which will result in an improved device and method of using the same, yet all of which will fall within the scope and spirit of the present invention as defined in the following claims. Accordingly, the invention is to be limited only by the following claims and their equivalents.

What is claimed is:

1. A method for positioning an ultrasound transducer of a fetal heart rate monitor along the skin of a pregnant patient comprising the steps of:
(a) placing the transducer on the skin of a patient;
(b) introducing an ultrasound signal into the patient toward the fetus and receiving an echo audible signal providing information as to the heart rate of a fetus within the patient;
(c) monitoring the strength of the echo audible signal; and
(d) converting the echo audible signal to a visual representation of the strength of the echo audible signal.

2. The method of claim 1 wherein the step of converting the audible signal includes the step of determining the levels of the average maximum signal strength and the average minimum signal strength.

3. The method of claim 2 wherein the step of converting the audible signal includes the step of calibrating a full scale audio level from the maximum and minimum determined signal strengths.

4. The method of claim 3 wherein the step of the step of converting the audible signal includes determining a predetermined percentage of the full scale.

5. The method of claim 1 further including the step of
(e) moving the transducer to a position along the skin of a patient based upon the visual representation of the strength of the echo audible signal monitored in step (c).

6. The method of claim 5 wherein the step of moving the transducer comprises moving the transducer based upon the strength of the echo audible signals.

7. A system for locating an ultrasound transducer along the skin of a pregnant patient comprising:
a system for exciting an ultrasound transducer to introduce ultrasound signals into a pregnant patient toward a fetus,
a receiver for receiving echo signals that return from the fetus;
a signal strength indicator for providing a visual indication of the strength of the echo signals returning from the fetus.

8. The system of claim 7 wherein the system further includes circuitry to convert the echo signals to a visible representation of the strength of the echo signals.

9. The system of claim 8 wherein the circuitry includes electronic means to determine an average maximum and an average minimum strength of the echo signals.

10. The system of claim 9 wherein the visual representation shows a real time echo signal relative to the average maximum and minimum strength echo signals.

11. The system of claim 8 wherein the signal strength indicator provides a visual representation of a number of bars of differing thickness.

12. A monitoring system for relocating an ultrasound transducer along the skin of a pregnant patient comprising:
an ultrasound transducer adapted to be located in contact with the abdominal skin of a patient,
a source of power to cause the ultrasound transducer to emit ultrasonic waves into a pregnant patient toward fetus,
a monitor adapted to receive return audio signals from the patient in the form of an audio envelope and to process those signals;
circuitry to convert the signals in the audio envelope into visible signals; and
a signal strength indicator for receiving the converted signals to provide a visual display of the strength of the audio signals returning from the patient.

13. The monitoring system of claim 12 wherein the signal strength indicator is located in the monitor.

14. The monitoring system of claim 12 wherein the signal strength indicator is located in the ultrasound transducer.

15. The monitoring system of claim 12 wherein the circuitry includes electronic means to determine an average maximum and an average minimum strength of the echo signals.

16. The monitoring system of claim 14 wherein the signal strength indicator displays the echo signals relative to the average maximum and minimum strength echo signals.
